(19) United States
(12) Patent Application Publication
(10) Pub. No.: US 2006/0074048 A1
(43) Pub. Date: Apr. 6, 2006

(54) Mixed esters of hyaluronic acid with retinoic and butyric acids

(76) Inventors: Alberto Perbellini, Verona (IT); Danila Coradini, Milano (IT)

Correspondence Address: Abelman Frayne & Schwab, 666 Third Avenue, 10th Floor, New York, NY, 10017-5621, US

(21) Appl. No.: 10/540,939

(22) PCT Filed: Dec. 22, 2003
(86) PCT No.: PCT/EP03/14732

§ 371 Date: Jun. 23, 2005

(30) Foreign Application Priority Data
Dec. 23, 2002 (IT) MI2002A002745

(51) Int. Cl. A61K 31/728; C08B 37/00

(52) U.S. Cl. 514/54; 536/53

(57) ABSTRACT

The present invention relates to mixed esters of hyaluronic acid, wherein the hydroxyl groups are partially esterified with retinoic and butyric acids. These mixed esters are characterized by specific degrees of esterification and by a high ratio between the degree of substitution with butyric acid and retinoic acid. They exhibit a high anti-proliferative activity associated with activation of cell differentiation, with consequent clinical relevance in the treatment of hyper-proliferative pathologies and in particular of solid and systemic tumors.

### FIELD OF INVENTION

The present invention relates to new anti-tumor drugs developed from polysaccharidic compounds endowed with the ability to deliver molecules specifically.

### STATE OF THE ART

Both Retinoic acid and Butyric acid are molecules used in the treatment of hyper-proliferative disorders, particularly in neoplasia.

Butyric Acid (hereafter referred to as BA) is one of the main short-chain fatty acids derived from the colonic fermentation of complex carbohydrates introduced with diet (Hill M J. Eur J Cancer Prevention 4:897-904, 1995; Cummings J H., Gut 22:763-779, 1981) and is physiologically present in millimolar concentrations in the colon, where it regulates the turnover of colonic epithelial cells by inducing cell differentiation and programmed cell death or apoptosis (Jass J R. Med Hypotheses 18:113-118, 1985; Guibaud N F, Gas N, Dupont M A, Valette A., J Cell Physiol 145:162-172, 1990). In addition to carrying out this important physiological role, BA is capable of inhibiting tumor growth according to experimental evidences obtained from a significant series of cell lines representative of the most common human solid tumors (Coradini D, Biffi A, Costa A et al., Cell Prolif 30:149-159, 1997; Coradini D, Pellizzaro C, Marimpietri D et al., Cell Prolif 33:139-146, 2000; Pellizzaro C, Coradini D, Abolafio G et al., Int J Cancer 91:658-664, 2001).

The mechanism of action underlying such activity seems to rely mainly on inhibition of the activity of histone de-acetylase enzymes (HDAC) which are important components of the complex that regulates gene transcription.

Retinoic acid (hereafter referred to as RA) is a monocarboxyilic polyunsaturated acid composed of 20 carbon atoms and represents the oxidized form of vitamin A, well known for its important biological functions in the process of vision, in maintenance of the cutaneous function, in haemopoiesis and in embryonic development (Lotan R, Biochem Biophys Acta 605:33-37, 1980; Dower D, Koeffler H P., Exp Cell Res 138:193-199, 1982). The mechanism of action of RA is strictly correlated with the presence of specific endocellular receptors (retinoic acid receptor, RAR), belonging to the class of ligand-dependent transcription factors, which regulate expression of genes responsible for cell differentiation. Because of their differentiating properties, RA and its derivatives have long been used in the treatment of several diseases characterized by abnormal cell proliferation, among which neoplasia (Hansen L A, Sigman C C, Andreola F, et al., Carcinogenesis 21:1271-279, 2000; DelUca L M, Kosa K, Andreola F., J Nutr Biochem 8:426-437, 1997).

Even though BA and RA are undoubtedly endowed with anti-proliferative properties, their clinical use is hampered by the rapid metabolism of the former and the toxicity of the latter, mainly due to an accumulation phenomenon that occurs at high doses.

It is possible to overcome these drawbacks by linking these active principles to opportunely selected carrier molecules which, in addition to stabilizing the molecule without altering its properties, can increase its bioavailability and can specifically deliver it to target cells, thereby increasing its pharmacological efficacy and reducing the side effects.

Among possible carrier molecules, hyaluronic acid (hereafter referred to as HA) turned out to be of special practical interest for selective delivery of biologically active molecules whose clinical use is limited by physico-chemical problems. In fact, hyaluronic acid is recognized by a specific membrane receptor, CD44, that is over-expressed in actively growing cells and particularly in tumor cells (Rudzki Z and Jothy S., J lin Pathol:Mol Pathol 50:57-71, 1997). From the chemical point of view, HA is a polysaccharide consisting of disaccharide units of glucuronic acid and N-acetylglucosamine, with molecular weight up to millions of Daltons, and with partially esterifiable hydroxyl groups.

Because of these esterifiable groups, it has been possible to link the BA to HA and obtain a pro-drug capable of chemically stabilizing the BA. This allows its selective delivery to cells over-expressing the CD44 receptor with a significant enhancement of the anti-proliferative effect (Coradini D, Pellizzaro C, Miglierini G, et al., Int J Cancer 81:411-416, 1999). Butyric esters of HA with high anti-proliferative activity have been described in the patent application WO98/23648. Polysaccharidic esters of Retinoic Acid have been described in the Italian Patent Application TS2001A000017.

Although of significant clinical interest, the synthesis of a pro-drug made of a single carrier molecule delivering two different active principles could present some disadvantages from the physico-chemical point of view. These are mainly due to steric hindrance by the first molecule added to the carrier, with consequent reduction of its reactivity with the second molecule to be bound, that would make the reaction more difficult and less controllable. Moreover, once it is accomplished, the double substitution could deeply modify the carrier structure influencing in unpredictable ways its solubility, permeability, and site-specificity properties. Finally, the release of two active molecules from the carrier might occur according to different and mutually competitive kinetics, with the result that the mere co-delivery of the two active principles would not guarantee their simultaneous bioavailability at the site of action.

Therefore the present the invention fulfils at the same time the needs for effectively carrying retinoic and butyric acids on one molecule and for solving problems inherent to the simultaneous presence of two active principles on the same carrier molecule.

### SUMMARY OF THE INVENTION

The present invention describes new mixed esters of hyaluronic acid with retinoic and butyric acids. These esters are characterized by a ratio between the degree of substitution with butyric acid and retinoic acid that is at least 6 and more preferably higher than 10. Furthermore, they are characterized by a degree of substitution with butyric acid that is preferably ranging from 0.05 to 1.0, even more preferably ranging from 0.1 to 0.35, and by a degree of substitution with retinoic acid ranging from 0.002 to 0.1, even more preferably ranging from 0.01 to 0.05.

Therefore mixed esters with these characteristics allow to reach a pharmacologically effective concentration of butyric and retinoic acids at the target site, potentiating their biological activity. Indeed, cytostatic and differentiating effects of the two active principles turned out to be even qualitatively and quantitatively greater than those obtained either by individual administration of the two acids or as association of the two mono-esters obtained by esterification with hyaluronic acid.

The cytostatic and differentiating activities of the above-said esters make possible to treat with greater effectiveness, as compared to current therapies, pathologies characterized by conditions of cell hyper-proliferation, including human solid and systemic tumors.

The esters of the invention give rise to solutions characterized by low viscosity and good solubility and therefore suitable for pharmaceutical compositions that can be easily administered to humans and animals.

Moreover, the present invention includes a process for preparation of the aforesaid esters consisting in the formation of an alcoholate of hyaluronic acid under particular reaction conditions: the alcoholate is first reacted with retinoic acid and then with butyric acid. Such procedure makes possible to easily obtain the desired degrees of substitution together with a controlled degradation of the native hyaluronic acid molecule within a medium-low molecular weight range.

### DESCRIPTION OF THE FIGURES

FIG. 1. Comparison between the anti-proliferative effect of scalar doses of retinoic-butyric mixed esters of hyaluronic acid and of butyric or retinoic monoesters in human mammary carcinoma cells (MCF7).

After 6 days of continuous treatment with scalar doses of the mixed ester of the invention (range: 2-0.0001 mg/ml), butyric mono-ester (range: 4-0.03 mg/ml) or retinoic mono-ester (range: 4-0.0001 mg/ml), the effect on cell growth was assessed by a colorimetric method (MTT) as percentage of inhibition compared to the control, represented by cells maintained in culture medium alone. For each concentration tested, the mixed ester exerts an anti-proliferative activity greater than the corresponding concentrations of both mono esters, thus suggesting a possible synergistic effect between the two active principles simultaneously present on the same carrier molecule.

Butyric monoester (HA-BA): -▪-; retinoic monoester (HA-RA): -●-; mixed ester of hyaluronic acid with retinoic acid and butyric acids (HA-BA/RA): -▾-.

FIG. 2. Effect of scalar doses of retinoic-butyric mixed ester of hyaluronic acid on growth of promyelo/monocytic cells (U937 and HL-60).

After 6 days of treatment with scalar doses of mixed ester (concentration range: 1-0.0001 mg/ml), the effect of the compound on cell growth was determined by a colorimetric method (MTT), as percentage of growth inhibition compared to the control (represented by cells cultured in medium alone). The growth of both cell lines was only partially inhibited. Even at 1 mg/ml, the highest concentration corresponding to 10⁻⁴ M RA and 2 mM BA (at which both active principles induce nearly 100% growth inhibition), it was observed only 40% inhibition in U937 and 48% in HL-60. This limited inhibition was observed despite the presence of the specific hyaluronic acid receptor CD44 that was checked before by flow cytometry. U937 cells: -▪-; HL60: -▪-.

FIG. 3. Cell cycle perturbations induced by the retinoic-butyric mixed ester of hyaluronic acid in the promyelolmonocytic cell line U937.

The evaluation was performed by flow cytometry (panels a, b, c) after 6 days of continuous treatment with mixed ester at 0.1 or 1 mg/ml concentrations and was compared with the effect on cell growth determined by a colorimetric method (MTT), described by the curve -●-.

Panel a): control (Go=42%; S=44%; b): mixed ester at 1 mg/ml concentration (Go=91%; S=5%); c): mixed ester at 0.1 mg/ml concentration (Go=94%; S=3%).

FIG. 4. Comparison between the effects of mixed esters of the invention and the active principle NaB on proliferation of the promyelocytic cell line U937.

In FIG. 4 is reported the growth inhibition induced by three double esters (HRE23, HRE24 and IS16) compared with NaBu (sodium butyrate). HRE23, HRE24, IS16: mixed esters according to the invention.

FIG. 5. Comparison between the effects of the mixed esters of the invention and the active principle on growth of the promyelocytic cell line U937.

It is reported the effect induced by three mixed esters (HRE23, HRE24 and IS16) compared with that of RA (retinoic acid). HRE23, HRE24, IS16: mixed esters according to the invention.

FIG. 6. Effect of retinoate-butyrate mixed ester and of the two active principles (butyric acid and retinoic acid) in the promyelo/monocytic cell line U937.

The effect on cell cycle and on cell proliferation has been assessed. The assessment has been performed by flow cytometry after 3 days of treatment with the mixed ester of the invention at 0.1 mg/ml concentration, or with butyric acid or retinoic acid at concentrations of 0.2 mM and 10⁻⁵ M, respectively. Cell cycle perturbations induced by these compounds are related to the effect on cell proliferation (ordinate axis) exerted by identical concentrations of the drugs and expressed as percentage of the control (CTR) represented by cells cultured in medium alone. Cytometric analysis indicates that the mixed ester induces maximal inhibition already after 3 days of treatment, with 72% of the cells blocked in the G_{0/1} phase of cycle. Instead BA at 0.2 mM concentration is totally unable to induce an anti-proliferative effect or a block of the cell cycle, and 10⁻⁵M RA significantly inhibits cell growth with a concomitant block in the G_{0/1} phase of the cell cycle. CTR: control. Panel a): (Go=45%; S=46%), panel b): (Go=44%; S=45%); panel c) (Go=43%; S=45%); panel d): (Go=72%; S=22%); panel e) (Go=41%; S=46%), panel f): (Go=94%; S=2%).

FIG. 7. Comparative evaluation of cytostatic and pro-differentiating effects of the compounds of the invention with the effects of pure or mono-ester forms of the active principles, administered individually or in combination to the promyelo/monocytic cell line U937.

The graph synthetically shows the effects on cell growth (expressed as percentage of cells blocked in the G_{0/1} phase of the cycle, % ordinate axis) and effects on differentiation (measured as percentage of cells expressing the CD11b antigen) induced by the various compounds administered individually or in association. The figure shows the assessment performed by flow cytometry after 3 days of treatment with the various compounds administered at a concentration corresponding to that in the mixed ester at a dose of 0.1 mg/ml.

FIG. 8. Comparison between the anti-proliferative effect of scalar doses of retinoic-butyric mixed esters of hyaluronic acid and of butyric or retinoic monoesters in the colon adenocarcinoma cell line HT29.

After 6 days of continuous treatment with scalar doses of mixed esters of the invention (range 1-0.0001 mg/ml), with butyric mono-ester (range 4-0.03 mg/ml) or with sodium butyrate (range 4-0.03 mg/ml), the effect on cell growth has been measured by a colorimetric method (MTT). The effect was expressed as percentage of inhibition versus the control represented by cells maintained in culture medium alone (ordinate: % inhibition). For each concentration tested, the mixed ester exerts an anti-proliferative activity greater than the corresponding concentration of mono-ester suggesting a potential synergistic effect between the two active principles present at the same time on the same carrier molecule. Butyric mono-ester (HA-BA): -▾-; Sodium butyrate (NaB) -●-; mixed ester of hyaluronic acid with butyric and retinoic acid (HBR): -▪-.

### DETAILED DESCRIPTION OF THE INVENTION

The invention describes new mixed esters of hyaluronic acid (HA) with retinoic and butyric acids, also named HBR for the purposes of the present invention. In these esters, the hydroxyl groups of hyaluronic acid are partially substituted with the acyl residues of retinoic acid (RA) and butyric acid (BA).

Said mixed esters are characterized by a higher content of butyric acid than retinoic acid according to a ratio between the degree of substitution (D.S.) with butyrate and with retynoate of at least 6 (DS BA/RA≧6) or, more preferably, higher than 10. Even more preferably, said esters have a degree of substitution with butyric acid ranging from 0.05 to 1.0, or even more preferably ranging from 0.1 to 0.35 and a degree of substitution with retinoic acid ranging from 0.002 to 0.1, or even more preferably ranging from 0.01 to 0.05.

Moreover, their molecular weight is preferably comprised within the range from 10.000 to 30.000 Daltons.

The term “molecular weight” indicates the average molecular weight (MW) of hyaluronic acid only, without considering the contribution of butyric and retinoic residues.

The term “degree of substitution” designates the number of esterified hydroxyl groups for each repetitive unit of hyaluronic acid (consisting of the dimer GlcNAc-GlcUA).

The term “retinoic acid” or “(RA)” designates all isomeric forms of this compound, hence both its natural form (with all the double bonds in trans-form), and all the other possible isomeric forms.

The mixed esters of HA described in the present invention simultaneously deliver pharmacologically effective amounts of retinoic and butyric acids to a specific target site of the cell. Therefore both pro-differentiating and cytostatic activities are qualitatively and quantitatively higher than those obtainable with the two acids administered individually or as association of mono-esters with hyaluronic acid.

In particular, the association of the two mmono-esters (HA-BA and HA-RA comparatively used) although producing the same effect on growth inhibition is not able to induce any significant effect on cell differentiation. This argues in favour of the remarkable potential of the mixed esters as compared to the active principles and to their mono-esters used separately or in association.

The anti-proliferative activity of the aforesaid mixed esters is exerted by a cytostatic action that is detectable as cell growth inhibition due to a block of cells in the G_{0/1} phase of cell cycle, followed by activation of cell differentiation or, at high concentrations, by cell death or apoptosis. The induction of cell differentiation can be monitored through the re-expression of membrane antigens specific for the tissue under consideration such as the activation of CD11a and CD11b surface antigens in pro-myelocytic cells.

Therefore the mixed esters of the present invention can block tumor growth and restore the process of cell maturation, or induce cell death in a dose-dependent manner in most tumors, thus exerting effects on both the control of disease and on its therapy. This makes their application useful for the treatment of both solid and systemic tumors, and can be particularly useful in the therapy of tumors of the promyelocytic lineage and specifically in the acute promyelocytic leukemia (APL), due to their strong pro-differentiating effect.

In the case of APL, it can be in fact hypothesized a direct mechanism dependent on the presence of the chimeric receptor characteristic of this disease. The particularly high biological activity of the esters of the invention can be attributed to their action at two levels: inhibition of HDAC activity (histon-deacetylases that inhibit normal differentiation) through the butyric residues and, at the same time, induction of differentiation through the retinoic residues present on the carrier molecule. In particular, the esters of the invention induce the re-expression of surface antigens CD11a and CD11b in cells of the pro-myelocytic lineage.

The presence of the specific hyaluronic acid receptor CD44 on tumor cells is instrumental for the mixed esters to exert these effects. The presence of this receptor has been largely shown in most human solid tumors including, for instance, breast, colon and lung adenocarcinomas and melanoma, as well as in some systemic tumors such as APL.

Due to their peculiar features, the mixed esters of the invention allow: (i) to achieve a suitable dosage and high bio-availability of both acids at the same site of action, (ii) to maintain the property of site-specificity of hyaluronic acid, used as carrier molecule, (iii) to achieve new pharmacological effects to improve the treatment of tumor pathologies, (iv) to obtain a drug with physico-chemical features that make it easy to administrate, (v) to reduce the toxicity of effective doses of retinoic acid.

Indeed, the activity of said compounds is not simply explained by the sum of the activities of the two active principles (RA and BA), but is rather explained by an unexpected qualitative and quantitative synergism. This is probably due to the simultaneous presence of the two active principles on the same carrier molecule, thus enabling a cellular interaction between RA and BA, unlike the individual esters supplied as mixture. In fact, it is revealing that the mixed esters of the invention potentiate the effect that is obtained with the simple association of retinoic and butyric acid or their mono-esters with hyaluronic acid.

The effects of the esters of the present invention on growth and differentiation of cells expressing the hyaluronic acid receptor (CD44) are totally unexpected, both in terms of behaviour of retinoic and butyric acids, separately or in physical mixture, and of simultaneous administration of single mono-esters of hyaluronic acid.

In fact, the cytometric analysis shows that the greatest inhibition of cell growth and a block of 72% cells in G_{0/1} phase of cycle are already obtained within only 3 days of treatment with the mixed ester of the invention. Instead the results obtained when the two active principles are separately added are those expected for the concentrations used. Indeed, BA at 0.2 mM concentration is totally ineffective in inducing an anti-proliferative effect or a cell cycle block, whereas retinoic acid at approximately 10⁻⁵M concentration (corresponding to a concentration of 0.1 mg/ml of the mixed ester) is cytotoxic, as it is well known.

Therefore, the mixed ester of the invention allows the administration of otherwise cytotoxic and therapeutically unsuitable doses of RA, and to obtain, at the same time, a pro-differentiating effect.

The greater biological activity observed with the mixed esters compared to the association of the two mono-esters can be at least partially ascribed to the absence of a mechanistic antagonism between the two mono-esters in their binding kinetic to the same receptor.

In synthesis, the applicants have observed that:

on a quantitative level, the mixed esters of HA with RA and BA, according to the present invention, show an anti-proliferative and cytotoxic or cytostatic effect that is dose-dependent and a pro-differentiating effect on cells derived from solid and systemic tumors with an activity significantly higher than that achieved by using the two individual acids, their physical mixture or their hyaluronic mono-esters;

on a qualitative level each of butyric and retinoic acids, their physical mixture and the respective hyaluronic monoester all show a pro-differentiating effect which is significantly lower than the effect on cell growth inhibition. It should be noticed that both anti-proliferative and pro-differentiating effects are extremely useful in anti-tumor therapy and that only the esters of the present invention are able to produce significant effects on both parameters.

In particular, using the U937 and the HL-60 cell line as in vitro system, it has been observed that, within the preferred range of degree of substitution for BA or RA, the mixed esters with a degree of substitution comprised from 6 to 10 for both BA and RA, show an IC₅₀ (the concentration producing 50% anti-proliferative effect) lower than 1 nM.

Another advantageous feature of the esters of the present invention is the controlled reduction of the molecular weight of HA. This feature reduces the steric hindrance of the final molecule (to the advantage for bioavailability), improves its solubility properties and reduces its viscosity in solution, making it more easily administrable to humans and animals. This is obtained without altering the site-specific delivering capacity of the native hyaluronic acid.

In fact, the hyaluronic esters of the present invention have features of optimal solubility and low viscosity, making them suitable for easy formulations and easy and harmless administration even intravenously or intra-muscularly.

In fact, the mixed esters of the invention can be prepared in the form of aqueous solution up to a concentration of at least 2 mg/ml.

An additional embodiment of the present invention entails the process to synthesize the aforesaid mixed esters of HA. This process is characterized by an esterification step with retinoic acid derivatives performed prior to esterification with butyric acid derivatives.

The process is characterized by the following steps, preferably in the order reported below:
i) formation of an alcoholate of hyaluronic acid;
ii) esterification of the alcoholate obtained in i) with retinoic acid derivatives to obtain a retinoic mono ester of hyaluronic acid;
iii) esterification of the mono esters obtained in ii) with butyric acid derivatives to obtain the aforesaid mixed ester of hyaluronic acid.

Preferably esterification with RA is carried out before the esterification with BA; moreover, it is even preferred that, prior to the esterification with RA, hyaluronic acid is transformed into the corresponding alcoholate according to the conditions indicated in point i).

The HA that is used as starting material can be used as such or in a salt form; preferred example of hyaluronic acid salts are the quaternary ammonium salts, such as the tetrabutyl ammonium salt. Hyaluronic acid is commercially available and has a molecular weight generally ranging between 10⁴ and 10⁷ Daltons.

The base employed in step i) for converting the hydroxyl groups of hyaluronic acid into their salts is preferably a quaternary ammonium hydroxide bearing C1-C5 alkyl radicals, such as tetrabutyl ammonium hydroxide. In this step a C1-C5 alcohol, preferably methanol, to dissolve hyaluronic acid.

The reaction is carried out at room temperature, on average for 2-3 hours. Under these conditions, hyaluronic acid undergoes a controlled reduction of molecular weight. At the same time, the hydroxyl groups are transformed into alcoholate, becoming activated for the subsequent reaction with retinoic acid. The alcoholate obtained in this way is then isolated from the reaction environment (eg. by removing the solvent by means of lyophilization). Hyaluronic acid is preferably used in the form of quaternary ammonium salt, while the esterification reaction according to point ii) is preferably carried out using retinoyl chloride as esterifying agent. To carry out step ii) the alcoholate from step i) is preferably dispersed in dimethylformamide and added to a retinoyl-chloride solution that was separately prepared before.

To carry out step iii) the following procedure is preferably followed: the retinoic monoester obtained in ii), preferably purified, is dispersed in dimethylformamide and triethylamine; butiryc anhydride and dimethylaminopyridine are then added to complete the esterification.

If preferred, the mixed ester can be further purified by washes, by dialysis, by passage through ionic exchange resins, etc.

The present invention further comprises any pharmaceutical composition containing the above-said mixed esters, individually or in association with other compounds or with pharmaceutically acceptable excipients, which are more suitable for the formulation. Examples of such pharmacological formulations are solutions, suspensions, soluble powders, tablets, granules, micro-capsules, soft or rigid capsules, coated tablets, suppositories, ovules, ointments, gel, etc.

The invention further comprises the use in therapy of the aforesaid esters and of their pharmacological compositions. According to this embodiment the invention comprises a therapeutic method to a patient in need of an antiproliferative and/or an antitumoral treatment, comprising administering to said subject a therapeutically effective amount of the mixed esters of the invention at therapeutically active doses.

In particular, the present invention allows the use of the above specified mixed esters for the preparation of antitumoral medicaments with an anti-proliferative and a pro-differentiating action, particularly useful in the therapy of systemic tumors such as acute promyelocytic leukemia.

More generally the anti-proliferative properties of the esters of the invention make them useful for the treatment of all the diseases characterized by cellular hyper-proliferaton, as for example inflammatory bowel diseases, Crohn's disease, ulcerative colitis, psoriasis, hyperkeratosis, prostatic hyperplasia, synovial cell proliferation.

The route of administration can be chosen, for example, among oral, intravenous, intramuscular, intra-peritoneal, rectal, intra-cavity, vaginal, trans-cutaneous, topic, etc.

The present invention will now be further explained by way of the following examples, without being limited thereto.

Experimental Part

Materials and Methods.

Cell Culture.

MCF7, U937, HL-60 and HT29 cell lines were purchased from the American Tissue Culture Collection (Rockville, Miss.). MCF7 cells were grown in DMEM/F12 supplemented with 5% FBS, while U937 and HL-60 were grown in RPMI-1640 supplemented with 10% (v/v) foetal bovine serum.

Cell Proliferation.

Approximately 1000 cells (MCF7 and HT29) and 500 cells (U937 and HL-60) per well were used in 96-well plates and were treated as indicated in the examples. At the end of the experiment, the anti-proliferative effect was measured with 3-(4,5-dimethylthiazol-2-yl)-2,5-diphenyltetrazolium bromide (MTT). The blank value, corresponding to wells containing all the components but cells, was subtracted from the absorbance values obtained.

Flow Cytometric Determination.

Expression of CD44, CD11a, CD11b, CD18 antigens was measured with specific murine monoclonal antibodies and secondary anti-mouse antibodies conjugated to FITC. After immunofluorescence, cells were incubated in a solution containing propidium iodide (PI) (5 mg/ml), Rnase (10 kU/ml), and Nonidet P40 (0.005%). The fluorescence relative to PI was measured using a FACScan flow cytometer (Becton Dickinson) equipped with an argon laser at 488 nm excitation wavelength and a 610 nm filter. The fluorescence signal was collected in linear and logaritmic modes. Data were evaluated using the LYSIS II software.

To evaluate cell cycle alterations, cells were re-suspended in a solution containing only propidium iodide, Rnase and Nonidet P40 and the fluorescence was measured using a FACScan flow cytometer.

### EXAMPLE 1

### Preparation of Mixed Ester with Higher Degree of Substitution

In accordance with the premises, several tests have been performed in order to obtain mixed butyric and retinoic esters of hyaluronic acid with different relative ratios of esterification with one or the other substituent. More specifically, we have chosen a sequential esterification approach where the first esterification step is with RA, because of its slower rate of reaction. In the different tests, the stoichiometric ratios of the reactants and also some operating conditions have been changed in order to obtain different degrees of esterification, although always the same general scheme of synthesis was maintained:
preparation of TBA-alcoholate of HA-TBA,
synthesis of retinoyl-chloride from retinoic acid and oxalyl-chloride,
esteriflcation of HA with retinoyl chloride,
esterification of HA-RET with butyric anhydride.

In detail, for this first synthesis 5.0 gr of HA-TBA have been dispersed in 5.2 ml of 40% TBA-OH and subsequently lyophilized. 2.4 g of retinoic acid have been dissolved in 20 ml of anhydrous N,N-DMF, stirred for three hours, in nitrogen atmosphere and sheltered from light. Separately, 1 ml of oxalyl chloride and 1 ml of N,N-DMF are added to 5 ml of diethyl-ether. After 10′, the retinoic acid solution was added drop-wise for approximately 20′, always in nitrogen atmosphere, sheltered from the light and with magnetic stirring. After that, the mixture was left stirring for 1 hour.

The lyophilized HA-TBA alcoholate was dispersed in 300 ml of N,N-DMF and the retinoyl chloride solution, previously prepared, was then added drop-wise to this solution in approximately 30′ and at RT.

At the end, the reaction was incubated for additional 17 hours, always under magnetic stirring. The system obtained in this way was then divided in two identical aliquots: in the first aliquot (A) the retinoylation was stopped by concentration under vacuum to ⅓ of the initial volume of the solution and poured onto 3 volumes of ethyl ether. A precipitate was obtained that was re-suspended in 100 ml of N,N-DMF to which 300 μl of TEA are added.

Both aliquots A and B are then treated with 640 μl of butyric anhydride and 2 g of DMAP, leaving the reaction stirring over-night sheltered from light and under nitrogen flow.

After addition of three volumes of diethyl ether, the aliquots were filtered into a Gouch filter, washed with 3×100 ml of diethyl ether and purified by dialysis in 2 l of distilled water.

The products purified in this way were then passed through a column of ion exchange resin in the sodium form and finally lyophilized. Yields and degrees of substitution of the two products HRE23 and HRE24 (synthetized from aliquot A and B) were respectively as follows: 0.8 g, d.s. retinoate 0.046, d.s. butyrate 0.94 (ratio DS-HB/DS-HR=20,43) for HRE23; 1.2 g, d.s. retinoate 0.066, d.s. butyrate 0.45 (ratio DSBut/DSRet=6.8) for HRE24.

### EXAMPLE 2

### Preparation of Mixed Ester with Lower Degree of Substitution

1) Retinoylation.

The previous test was repeated using half the amount of retinoic acid and butyric anhydride.

(a) Preparation of the TBA-alcoholate of HA-TBA.

5.0 g of HA-TBA were solubilized in 20 ml of Methanol and 2.7 ml of a 40% (w/v) aqueous solution of TBA-OH, leaving the reaction for one night under magnetic stirring. The solvent was evaporated at reduced pressure and the product was lyophilized.

(b) Synthesis of Retinoyl Chloride.

Under nitrogen flow and sheltered from light, 0.5 ml of N,N-DMF and 0.5 ml of oxalyl chloride in 3 ml of diethyl-ether were added to a solution containing 1.25 g of retinoic acid in 10 ml of N,N-DMF by means of a dropping funnel (flow rate of 0.5 ml/min) and the and the mixture was left to react for one hour.

c) Synthesis of Retinoate Ester.

The previously prepared alcoholate was dissolved in 300 ml of N,N-DMF. The retinoyl chloride solution was then added by means of a dropping funnel (flow rate 1 ml/min) and the mixture was left to react for 22 hours.

2) Butyrilation

4.0 g of DMAP and 1.3 ml of butyric anhydride were resupended in 30 ml N,N-DMF.

The solution was added to the final system for retinoylation by a dropping funnel at 1 mL/min and left to react for 21 hours under mechanical stirring and sheltered from light.

The solution was concentrated under reduced pressure to ¼ of the initial volume. The product was recovered by precipitation in 6 volumes of diethyl-ether filtering under vacuum and washing at first with diethylether and then with acetone The solid pellet was then re-suspended in distilled water and dyalized 4 times against 20 l of distilled water. The purified solution was twice passed through an ionic exchange Amberlite IR 120 resin in sodium form (flow rate 5 ml/min) and was finally lyophilized.

2.74 g of mixed ester were obtained (IS-16 lot). DS_{BUT}=0.255; DS_{RET}=0.019.

### EXAMPLE 3

### Preparation of the Mixed Ester on Industrial Scale

The test in example 2 has been repeated on a scale 10/1.

1) Retinoylation.

50 gr of HA-TBA were solubilized in 300 ml of methanol and treated with 27 ml of 40% TBA-OH solution.

5 ml of N,N-DMF and 5 ml of oxalyl chloride were added to 30 ml of diethyl-ether and a solution containing 12.5 g of retinoic acid in 100 ml of N,N-DMF was added. 2.5 l of alcoholate solution in N,N-DMF was added drop-wise to this solution during 21 hours at room temperature.

2) Butyrilation.

To the retinoate so obtained, 20 g of DMAP and 13.2 ml of butyric anhydride were added. The reaction was left to proceed for 24 hours and the solvent was evaporated under vacuum. Then the compound was precipitated with ether and the aqueous solution was dyalized. The compound was passed through a resin in sodium form. The compound was then lyophilized and the yield calculated. 7 grams mixed ester were obtained with a DS But=0.325 and a DS Ret=0.015

### EXAMPLE 4

### Effect of the Mixed Esters on a Mammary Carcinoma Cell Line (MCF7) and on Promyelo/Monocytic Tumor Cell Lines (HL60 and U937).

The selected experimental model consisted of a human mammary carcinoma cell line (MCF7) as example of solid tumor. The effects induced by the mixed ester were compared with those obtained with the two active principles either in free form or bound to HA as monoesters. The presence of the CD44 receptor, specific for HA, was previously demonstrated (Coradini D, Pellizzaro C, Miglierini G, et al. Int J Cancer 81:411-416, 1999).

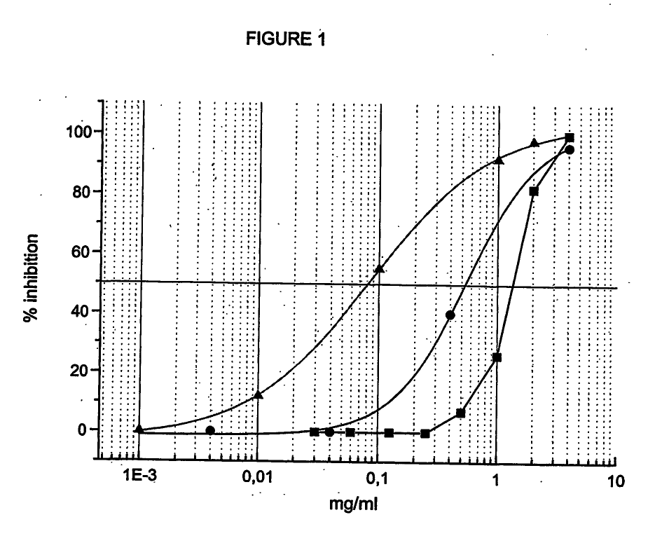

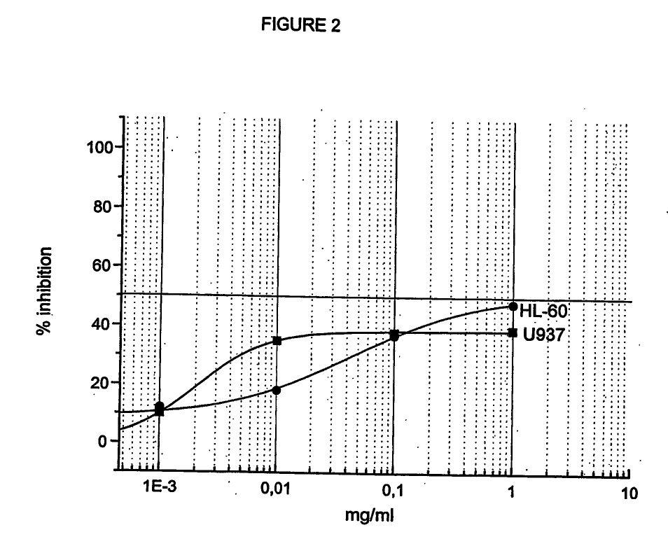

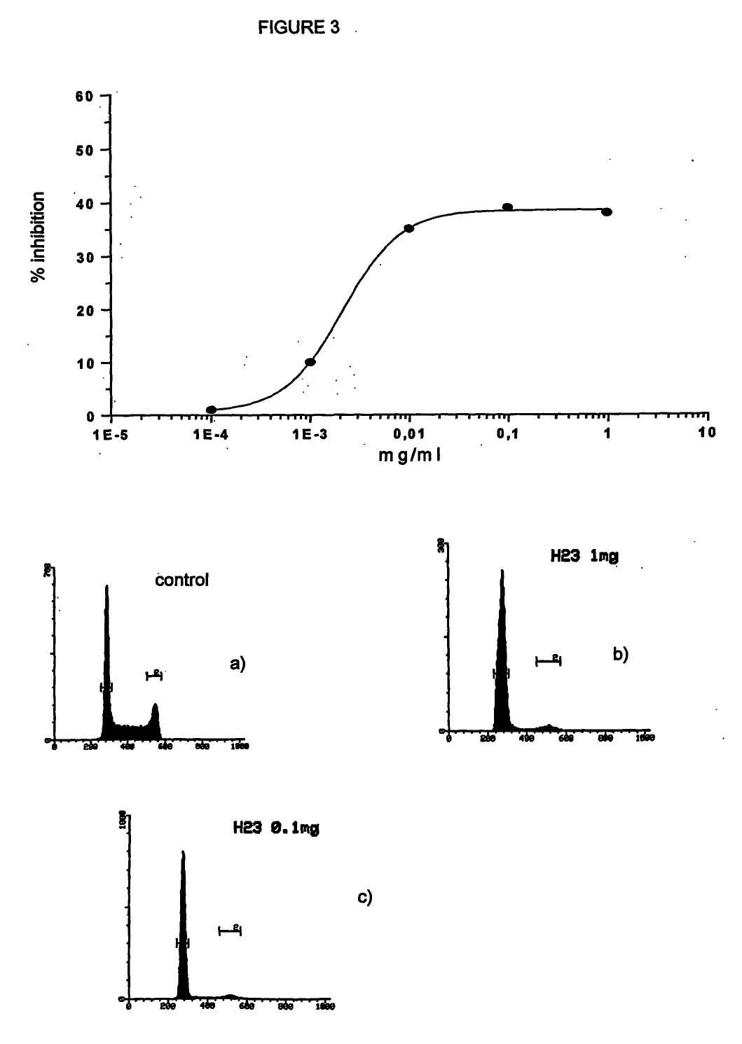

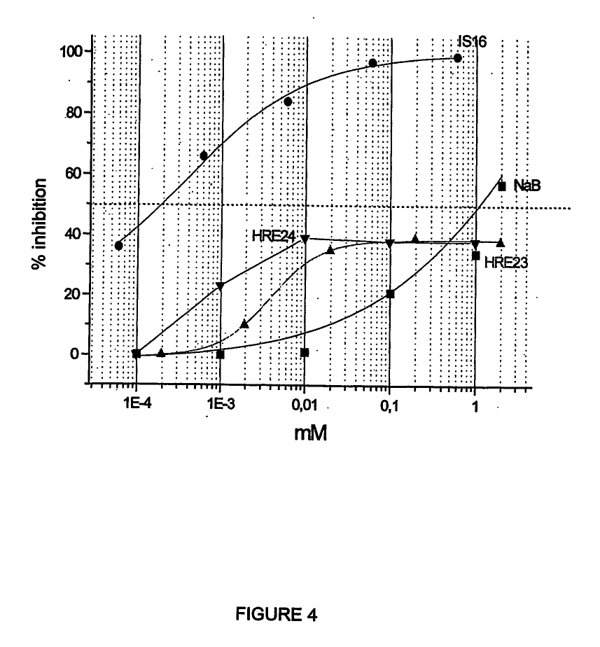

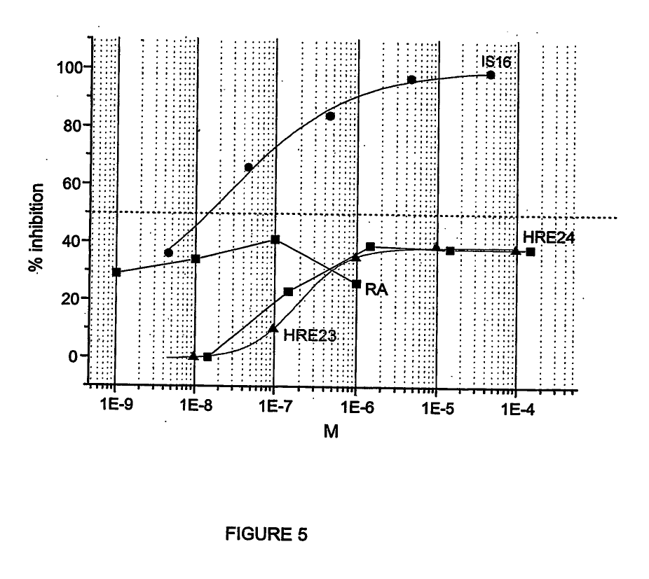

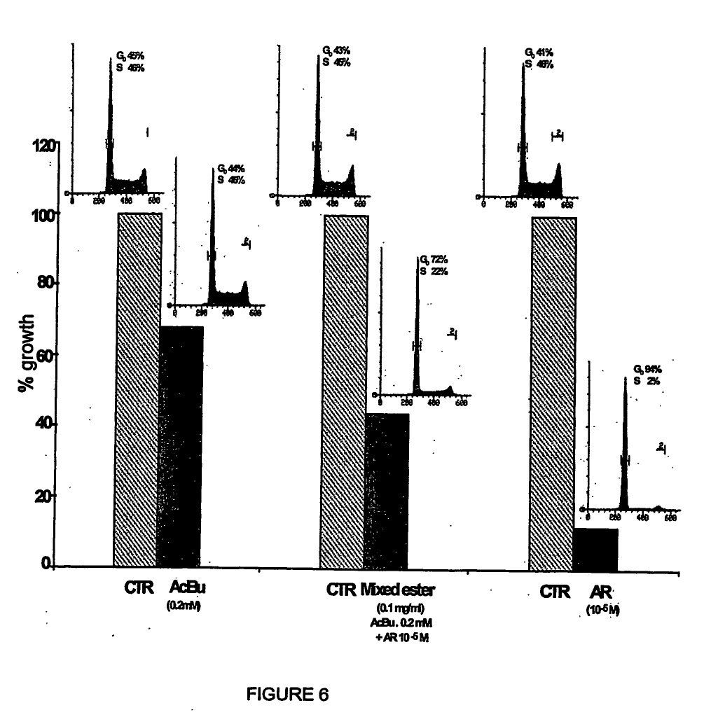

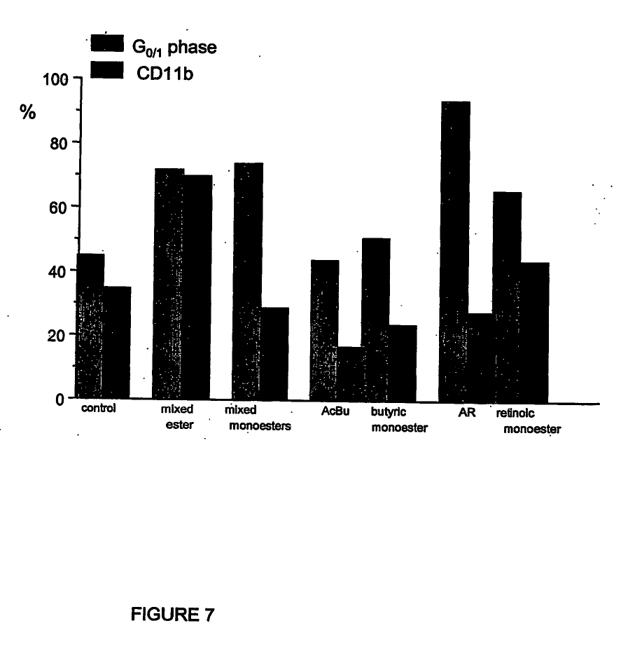

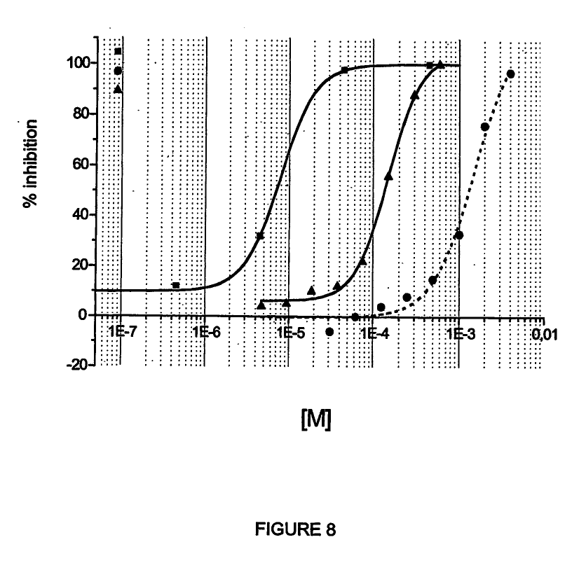

FIG. 1 shows that, after 6 days of treatment, scalar doses of mixed esters (range 2-0.0001 mg/ml) exert higher anti-proliferative activity than corresponding concentrations of both mono-esters of butyric acid and retinoic acid (range 4-0.0001 mg/ml). This suggests a possible synergistic effect of the two active principles simultaneously present on the same carrier molecule. Thus, for instance, whereas 1.4 mg/ml of butyric mono-ester and 0.5 mg/ml of retinoic mono esters were necessary to obtain 50% inhibition of cell growth, a concentration of 0.08 mg/ml of mixed esters was sufficient to obtain the same effect.

The promyelo-monocyitic HL-60 and U937 cell lines were chosen as examples of human systemic tumor. These cell lines undergo specific granulocytic (HL-60) or monocytic/macrophagic (U937) differentiation in presence of differentiating compounds such as retinoic acid. The mixed esters experimentally tested were characterized by the degrees of substitution specified in table 1.

TABLE 1

| Name | DS But | DS Ret | DS But/DS Ret |
|---|---|---|---|
| HRE23 (Ex.1) | 0.94 | 0.046 | 20.43 |
| HRE24 (Ex.1) | 0.45 | 0.066 | 6.6 |
| IS16 (Ex. 2) | 0.255 | 0.019 | 13.14 |

The effects induced by various mixed esters were compared with those of the two free active principles and with those obtained in some experiments with single mono esters of hyaluronic acid (for instance those reported in FIGS. 1 and 7).

As shown in FIG. 2, after 6 days of treatment with scalar doses of mixed ester (0.0001-1 mg/ml), growth of both cell lines was only partially inhibited. In fact, inhibition was 40% for U937 and 48% for HL-60 even in presence of the highest administered concentration of 1 mg/ml, corresponding to 10⁻⁴ M of RA and 2 mM of BA (at these concentrations both active principles induce nearly 100% of growth inhibition). This was observed despite the presence of the specific hyaluronic acid receptor CD44, checked by flow cytometry. Surprisingly, this modest anti-proliferative effect was associated with a significant increase in the percentage of cells blocked in the G_{0/1} phase of the cell cycle. In fact, as evidentiated by flow cytometry (FIG. 3), U937 cells treated with the mixed ester were almost totally blocked in the G_{0/1}/phase of the cell cycle even at a concentration of 0.1 mg/ml, corresponding to 10⁻⁵ M of RA and 0.2 mM of BA.

In FIGS. 4 and 5 it is shown a cytotoxic activity that is way above that of the active principle alone, both for butyric acid (FIG. 4) and retinoic acid (FIG. 5). In particular, the compound IS16 proved to be particularly active in that it showed an IC₅₀ of approximately 1×10⁻⁴ mM.

### EXAMPLE 5

### Effect of the Mixed Esters on the Expression of Differentiation Antigens

To check whether the block of cell growth was accompanied by activation of differentiation, the selected experimental model consists of the U937 promyelo/monocytic cell line, in which the effect of mixed esters on a series of myelocyte/macrophage specific differentiation markers (CD11a, CD11b and CD18) has been studied. The resulting effect has been directly compared with that of the two active principles. Since the growth tests showed that the mixed ester induces maximal effect already at a concentration of 0.1 mg/ml, experiments have been carried out at this same concentration. The results have been compared with those obtained with butyric and retinoic acids at the same concentrations as in the mixed ester (respectively 0.2 mM and 10⁵M). Since the time of treatment was reduced to 3 days in these experiments, the effects on growth and cell cycle were also measured.

FIG. 6 indicates that, after 3 days of treatment, the mixed ester already induces the highest inhibition, with 72% of cells blocked in G_{0/1} phase of the cell cycle. The results obtained with the two active principles, individually added, were as expected for the concentrations that have been administered. In fact, BA at 0.2 mM concentration was totally ineffective in inducing both an anti-proliferative effect and a cell cycle block, whereas RA at 10⁻⁵ M concentration, known to be cytotoxic and thus therapeutically unsuitable, significantly reduces cell growth with a concomitant cell block in the G_{0/1} phase of the cycle. The cytotoxic effect of RA at this concentration was confirmed by alterations of cell morphology observed under the microscope.

In table 2 are indicated the percentages of expression measured by FACS of some membrane antigens in U937 cells treated with the esters of the invention and with the two non-esterified active principles. For each treatment the control has been repeated and is then indicated by the respective superscript.

The evaluation was performed by flow cytometry after that the promyelo/monocytic U937 cell line was treated for 3 days with mixed ester at 0.1 mg/ml concentration, butyric acid at 0.2 mM concentration or retinoic acid at 10⁻⁵M concentration. Human lymphocytes were used as negative control for the expression of the antigen under examination.

TABLE 2

| Antigens | Controls (%) | butyric acid^{b} | retinoic acid^{c} | mixed ester^{a} |
|---|---|---|---|---|
| CD44 | 97^{a}, 98^{b}, 98^{c} | 91% | 97% | 91% |
| CD11a | 66^{a}, 76^{b}, 74^{c} | 76% | 91% | 95% |
| CD11b | 41^{a}, 27^{b}, 29^{c} | 17% | 28% | 70% |
| CD18 | 81^{a}, 77^{b}, 71^{c} | 82% | 83% | 82% |

By looking at table 2 it is clear that the growth inhibition induced by the mixed ester is associated with increased expression of CD11a and CD11b. The membrane antigen CD18, already constitutively expressed in high percentage, was not further induced.

In contrast, BA at the concentration tested (0.2 mM) does not change the expression of any marker, while RA induces the expression of CD11a only, differently from what was observed for the mixed ester.

Therefore, the arrest of proliferation induced by the mixed ester, shown in FIG. 6, was accompanied by increased expression of CD11a and CD11b, as shown in table 2, which are differentiation markers for the promyelocytic U937 cell line. Instead the CD18 antigen, already constitutively expressed in a high percentage of cells, was not further induced.

Even though it was involved in the uptake and internalization process, CD44 expression was not modified by administration of the compounds of the invention. This was probably due to the rapid turnover of the receptor that allows its internalization and reexpression on the cell membrane, and to the positive feedback induced by HA. This observation supports the possibility that a continuous treatment with the mixed ester would not reduce its uptake because of receptor depletion.

In addition to its individual component active principles, the effect of the mixed ester has been compared with the two mono-esters, separately or in association, that have been administered at concentrations corresponding to those in the mixed ester. It was observed that only the mixed ester was able at the same time to block cell growth (expressed as percentage of cells in the G_{0/1} phase of the cycle) and to promote cell differentiation (expressed as percentage of cells expressing CD11b antigen). In fact, as shown in FIG. 7, the two mono-esters in association have a similar effect on the block of cell growth but were unable to induce any differentiation. This accounts for the remarkable potential of the mixed ester as compared to single active principles, their mono-esters and association of mono-esters.

### EXAMPLE 6

### Effect of the Mixed Esters on a Colon Adenocarcinoma Cell Line

The encouraging results obtained with the previous cell lines, mainly consisting of a growth arrest associated with re-expression of promyelocytic differentiation-specific membrane antigens, suggested to continue with experiments on a series of cell lines derived from human solid tumors, known in the literature to express retinoic acid receptors (RARα/RARβ).

Particularly interesting were the results obtained with the colon adenocarcinoma cell line HT29. In fact, as can be inferred from FIG. 8, the mixed ester has an inhibitory effect definitely greater than the mono-ester. This is shown by its IC₅₀ value that is 23-fold and 230-fold lower than butyric mono-ester and sodium butyrate, respectively, suggesting in this cell line a synergistic inhibitory effect due to the simultaneous presence of retinoic and butyric residues.

The results shown in FIG. 8, and the observation of a higher responsiveness to double ester than to mono ester in a myelo-monoblastic line (U937) and also in solid tumor-derived cell lines (e.g. HT29), suggest that the double ester is particularly useful for treatment of tumors presenting both molecular targets, retinoic acid receptor and histon-deacetylase enzymes.

### EXAMPLE 7

### Evaluation of Acute Toxicity, with Determination of LD50 and of Sub-Acute Toxicity of the Mixed Esters in Mouse

For experiments on toxicity, a mixed butyric-retinoic ester of hyaluronic acid was used, whose main physico-chemical characteristics are shown in table 3.

TABLE 3
Physico-chemical characteristics of the mixed
butyric-retinoic ester of hyaluronic acid.

| | DS | | % (w/w)_{free} | | % (w/w)_{bound} | | |
|---|---|---|---|---|---|---|---|
| Derivate | BA | RA | BA | RA | BA | RA | M_{dimer} |
| HBR3 | 0.255 | 0.0195 | 5.30 | 1.38 | 5.24 | 1.37 | 424.41 |

Animals were treated according to the European Directive on “Guidelines for maintenance and use of laboratory animals”, adopted by the Italian legislation with the DDL n°116 of the 21-2-1992

Systemic toxicity of the mixed ester according to the invention (HBR—hyaluronic acid doubly esterified with butyric acid and retinoic acid) was determined after intraperitoneal and subcutaneous administration. The choice of these administration routes was justified by the constraints imposed by the characteristics of solubility of the compound, which condition the volumes that can be administered to an animal. The LD50 turned out to be higher than 500 μl/mouse (equivalent to 12 μmol/mouse).

Groups of five female Swiss mice, weighting 20-22 g, were treated according to the indicated administration route with the maximal administrable dose of 12 μmol/mouse of mixed ester (HBR). The average variation of body weight ( [symbol] ) is expressed as weight variation of the measurements obtained 24 hours before and 6 days after treatment. After treatment, the animals were kept under observation for a period of 30 days and no death was observed at the dose that was used. Body weight variations within the considered period were not significantly different from those of untreated animals. Data for acute toxicity are summarized in table 4.

TABLE 4
Acute toxicity of in vivo treatment with HBR administered
intraperitoneally or subcutaneously.

| Dose | Administration | Lethality | Variation of body |
|---|---|---|---|
| (μmol/mouse) | Route | death n./total n. | weight (g) [symbol] |
| HBR (12 μmoles) | Intraperitoneal | 0/5 | +0.80 |
| HBR (12 μmoles) | Subcutaneous | 0/5 | +0.20 |

[symbol]

Average body weight variations measured at the beginning and at the end of the treatment.

Considering the same two administration routes, the sub-acute toxicity of the mixed ester was also evaluated. HBR turned out to be not toxic when administered subcutaneously or intraperitoneally, for a maximum time of 7 consecutive days, at a dose of 400 μl/mouse (corresponding to 9.6 μmol/mouse).

Groups of five female Swiss mice, weighting 20-22 g were treated subcutaneously and intraperitoneally with 400 μl/mouse of HBR for 7 consecutive days. After treatment, animals were kept under observation for a period of 30 days. Body weight variations were not significantly different from untreated animals. No death was observed at any concentration that was used. The data concerning acute toxicity are summarized in table 5.

TABLE 5
Subacute toxicity of in vivo HBR treatment subcutaneously or
intraperitoneally administered.

| | Administration | | |
|---|---|---|---|
| Dose | Route | Lethality | Body weight |
| (μl/mouse/day) | (x days) | Death n./total n. | variations (g) [symbol] |
| HBR 400 μl | Subcutaneous | 0/5 | +0.40 |
| | (x 7) | | |
| HBR 400 μl | Intraperitoneal | 0/5 | +0.60 |
| | (x 7) | | |

[symbol]

Average body weight variation measured at the beginning and the end of the treatment.

### EXAMPLE 8

### Evaluation of the Pharmacological Activity of the Mixed Esters in Mouse

Evaluation of the pharmacological activity of the mixed esters of the invention in the experimental model of TLX5 lymphoma was then performed with the same compound used for toxicological experiments, the characteristics of which are reported in table 3. As summarized in table 6, intraperitoneal treatment with HBR for 7 consecutive days significantly reduces the number of tumor cells present in the peritoneum, in animals sacrificed 8 days after implantation.

TABLE 6
Effects of in vivo treatment with HBR on survival time and
on tumor cell number in TLX5 lymphoma-bearing mice.

| Treatment | Survival [symbol] | peritoneal ascites ♦ | Reduction |
|---|---|---|---|
| (μl/mouse) | (days) | (cell number ×10⁶) | % |
| Controls | 10.33 ± 0.9 | 920.6 ± 193.3 | |
| HBR (200) | 10.75 ± 0.8 | 679.9 ± 29.3 | 26.1% |
| HBR (400) | 12.5 ± 1.5 | 252.6 ± 122.5* | 72.6% |

The experiment was performed as follows: at day 0, groups of 8 male CBA/Lac mice were intraperitoneally injected with 100.000 TLX5 lymphoma cells. Twenty-four hours after tumor implantation, mice were then treated with HBR at a concentration of 6 or 12 μmol/mouse (200 μl or 400 μl intraperitoneum) for 7 consecutive days. The survival time was evaluated for 5 animals in each group ( [symbol] ) and the remaining animals were sacrificed at day 7, in order to count peritoneal tumor cells (♦).

Data are expressed as mean±s.e.; mean values labeled with the symbol (*) are significantly different from controls (p<0.05; T-test).

The anti-neoplastic effect, assessed by cell count in peritoneal ascites, was already evident at the lowest dose and becomes significantly different from controls at the highest dose used (400 μl/mouse/day). Although the survival time was increased, it was not significantly different from the controls. This was already observed in previous experiments performed in the same model and under same operating conditions, with butyrate mono-ester of hyaluronic acid (HB), as shown in the results shown in Table 7.

TABLE 7
Effects of repeated in vivo treatment with HA-BA on survival time
and on tumor cell number in mice with TLX5 lymphoma.

| Treatment | | | |
|---|---|---|---|
| (μmol/ | Survival [symbol] | Peritoneal ascites ♦ | Reduction |
| mouse) | (days) | (cell number ×10⁶) | % |
| Controls | 9.6 ± 0.2 | 689.0 ± 126.0 | |
| HB (250) | 10.4 ± 0.8 | 471.3 ± 48.1* | 31.6% |
| HB (500) | 11.6 ± 0.6 | 216.3 ± 48.5* | 68.6% |

Comparing tables 6 and 7, it can be noticed that the results on cell count reduction obtained with the highest doses of the mixed ester (⅕ of the corresponding dose of mono-ester) were better than those obtained with the mono-ester. In this experimental model, where the tumor type is particularly aggressive, a 70% reduction of cell load is anyway insufficient to increase significantly the survival time, since it corresponds to a life time increase of only 2 days for treated versus control animals. This time is insufficient to determine the minimal threshold of 25% increment of survival versus controls.

The TLX5 lymphoma was used as experimental model for screening different molecules and was normally used for evaluation of highly cytotoxic chemotherapeutic drugs exactly because of its extreme aggressiveness. In this model, a cell count reduction greater than 70% for drugs with anti-proliferative and/or pro-differentiating mechanisms of action, like HB and the HBR, indicate a remarkable anti-tumor activity.

It is predictable that the anti-tumor efficacy of HBR on other tumors, particularly those localized in the liver, where hyaluronic acid glucoconjugates preferentially accumulate, will turn out to be as high as the reported efficacy in the treatment of the hepatic metastases of melanoma. In the latter case, treatment with HA-BA resulted in complete recovery in more than 85% of the animals.

Moreover, it is worth noticing that the anti-tumor effect of the mixed esters of butyric and retinoic acids is independent from the histological origin of the tumor. These compounds act on mammary tumors as well as on lung tumors or melanomas, suggesting a wide spectrum of action of HBR in the oncological field.

1-22. (canceled)

23. Mixed ester of hyaluronic acid wherein hydroxyl groups of hyaluronic acid are partially esterified with retinoic acid and butyric acid molecules, characterized in that the ratio between the degree of substitution of said hyaluronic with butyric acid and the degree of substitution with retinoic acid is at least 6.

24. The ester according to claim 23, wherein said ratio is at least 10.

25. The ester according to claim 23, wherein the degree of substitution of hyaluronic acid with butyric acid ranges between 0.05 and 1.0 and the degree of substitution with retinoic acid ranges from 0.002 to 0.1.

26. The ester according to claim 25, wherein the degree of substitution with butyric acid ranges between 0.1 and 0.35 and the degree of substitution with retinoic acid ranges between 0.01 and 0.05.

27. The ester according to claim 23, wherein the average molecular weight (MW) of hyaluronic acid ranges from 10,000 to 30,000 Da.

28. A pharmaceutical composition comprising as the active ingredient the mixed ester of hyaluronic acid of claim 23.

29. A process for preparation of a mixed ester according to claim 23, comprising an esterification step of hyaluronic acid with retinoic acid carried out before the esterification with butyric acid derivatives.

30. The process according to claim 29, comprising the following steps:
i) formation of an alcoholate of hyaluronic acid;
ii) esterification of the alcoholate obtained in i) with retinoic acid derivatives to obtain a retinoic monoester of hyaluronic acid;
iii) esterification of the monoester obtained in ii) with butyric acid derivatives to obtain the aforesaid mixed ester of hyaluronic acid.

31. The process according to claim 30, wherein the hyaluronic acid is used as a quaternary ammonium salt.

32. The process according to claim 30, wherein in step i) the pH of the reaction environment is at least 13.

33. The process according to claim 30, wherein the esterification reaction according to step ii) is carried out using retinoyl chloride as esterifying agent.

34. The process according to claim 30, wherein the esterification reaction as in point iii) is carried out using butyric anhydride as esterifying agent.

35. The mixed ester of hyaluronic acid having both a cytostatic and a pro-differentiating activity obtainable according to the process of claim 29.

36. Pharmaceutical composition, carrying as the active principle at least one of the esters according to claim 35 in combination with pharmacologically acceptable excipients and/or diluents.

37. Pharmaceutical composition according to claim 28, in the form of solution, suspension, soluble powder, granule, soft or rigid capsule, micro-capsule, tablet, coated tablet, suppositories, ovuli, ointment, gel.

38. A method to induce the re-expression of surface antigenes CD11a and CD11b on a cell by using the mixed ester according to claim 23.

39. A therapeutic method for the treatment of diseases characterized by cellular hyper-proliferation in a subject in need of such a treatment, comprising administering to said subject a therapeutically effective amount of the antiproliferative and prodifferentiating mixed esters of the invention according to claim 23 or 35 at therapeutically active doses.

40. The method according to claim 39 wherein the diseases are inflammatory disease and tumors.

41. The method according to claim 40 wherein said inflammatory diseases are: inflammatory bowel diseases, Crohn's disease, ulcerative colitis, psoriasis, hyperkeratosis, prostatic hyperplasia, synovial cell proliferation.

42. The method according to claim 40 wherein said tumors are solid or systemic tumors.

43. The method according to claim 42 wherein said systemic tumors are: acute leukemia, acute promyelocytic leukemia, lymphomas, histiocytomas.
